(19) United States
(12) Patent Application Publication
(10) Pub. No.: US 2017/0327858 A1
(43) Pub. Date: Nov. 16, 2017

(54) EXTRACTS OF WHOLE STILLAGE AND OTHER BIOMASS AND METHODS THEREOF

(71) Applicant: Lakeview Nutrition LLC, Chicago, IL (US)

(72) Inventors: Dejian Huang, Chapel Hill, NC (US); Jim Galvin, Chicago, IL (US); Eamonn Byrne, Chicago, IL (US)

(21) Appl. No.: 15/528,822

(22) PCT Filed: Dec. 1, 2015
(86) PCT No.: PCT/US2015/063098

§ 371 Date: May 23, 2017

Related U.S. Application Data
(60) Provisional application No. 62/086,570, filed on Dec. 2, 2014.

(51) Int. Cl. C12P 19/30; C11B 1/02; C07K 14/425; C11B 1/10; C12F 3/10; C12P 19/00; C11B 1/00

(52) U.S. Cl. CPC C12P 19/30; C11B 1/10; C11B 1/025; C12F 3/10; C07K 14/425; C11B 1/00; C12P 19/00

(57) ABSTRACT

The invention provides novel and improved methods that allow effective capture of valuable active ingredients in biomass such as whole stillage or thin stillage at cost-effective commercial scale. The invention also provides novel compositions of active ingredients with unique properties (e.g., nutritional values and enhanced bioavailability).

### PRIORITY CLAIMS AND RELATED PATENT APPLICATIONS

This application claims the benefit of priority from U.S. Provisional Application Ser. No. 62/086,570, filed on Dec. 2, 2014, the entire content of which is incorporated herein by reference in its entirety.

### TECHNICAL FIELDS OF THE INVENTION

The invention generally relates to technologies for utilization of biomass. More particularly, the invention provides novel processes that enable efficient, large scale capture of many valuable components from biomass (e.g., whole stillage, thin stillage, syrup, beer, wet distillers grain with solubles), and compositions and uses thereof.

### BACKGROUND OF THE INVENTION

Whole stillage and thin stillage are by-products of the distillery process. Ethanol is produced on the industrial scale by fermentation of biomass such as corn, wheat, barley, rye and grain sorghum. The whole grain is ground into a course powder. An aqueous slurry of yeast cells and residuals from the ground grain remaining after fermentation pass through a stripper where the ethanol is recovered. The non-volatile components then leave as a product referred to as “whole stillage”. Whole stillage contains both dissolved and unfermented components and nondistillable microbial by-products, is rich in nutrients, fiber, oil, protein, lipids and yeast and has traditionally been incorporated into animal feed rations. Whole stillage is usually dewatered and separated into a liquid fraction (referred to as “thin stillage”) and a solids fraction (referred to as “wet grains” or “wet cake”).

Nutrient compositions of whole and thin stillage depend on the sources and quality of grain used and the specific processes that generated them. Most of the ethanol produced in the U.S. is made from corn. Because corn contains about two-thirds starch and most starch is converted to ethanol during fermentation, abundant nutrients (e.g., protein, fat, fiber, ash and phosphorus) remain as components of whole and thin stillage from fermentation of corn. There can be large variations in the nutrient content and quality produced in different plants. Besides corn, wheat, barley, rye and sorghum (milo) may also be used in alcohol production. Whole stillage and thin stillage from wheat have much higher protein and much lower fat content than distillers products from corn and sorghum.

A continuing challenge has been to better utilize the large amounts of biomass generated from ethanol plants and brewers. Many useful components in whole and thin stillage have not been efficiently captured at industrial scale. An urgent need exists for novel and improved tools to better utilize these valuable components in biomass, to reduce production cost of fuel ethanol, and to minimize the environmental burden.

### SUMMARY OF THE INVENTION

The invention is based, in part, on the discovery of novel and improved technologies that allow the efficient capture of valuable active ingredients from biomass such as whole stillage and thin stillage, at cost-effective commercial scale. Active ingredients that can be captured include, for example, vitamins, flavonoids, carotenoids, tocopherols, and lipophilic phenolics, phenolic acids and nucleotides. The extract compositions of the invention present a set of active ingredients in unique proportions as such as enhanced nutritional values and bioavailability.

In one aspect, the invention generally relates to a process for extracting one or more active ingredients from a biomass feedstock. The process includes: (a) contacting a biomass feedstock selected from a whole stillage or thin stillage with an enzyme capable of causing the biomass feedstock to release one or more nucleotides, thereby forming a mixture of a solid phase and an aqueous phase comprising one or more nucleotides; (b) separating the solid phase from the aqueous phase; (c) removing water from the aqueous phase to form a first product comprising one or more nucleotides; (d) contacting the solid phase with a solvent under a condition and for a time sufficient to extract the one or more active ingredients from the solid phase into the solvent, thereby forming a solid residual biomass phase and a liquid phase comprising the solvent and the extracted one or more active ingredients; (e) separating the solid residual biomass phase and the liquid phase comprising the solvent and one or more active ingredients; (f) selectively extract the liquid phase to yield a second product; (g) selectively extract the liquid phase to yield a third product; and (h) selectively extract the solid residual biomass phase to yield a fourth product.

In another aspect, the invention generally relates to a process for extracting one or more active ingredients from a biomass feedstock. The process includes: (a) contacting a biomass feedstock selected from a whole stillage with an enzyme capable of causing the biomass feedstock to release one or more nucleotides, thereby forming a mixture of a solid phase and an aqueous phase comprising one or more nucleotides; (b) adding lipophilic solvent, preferable ethyl acetate to extract the lipid soluble components from the mixture, (c) separating oil phase, water phase, and solid phase; (d) removing water from the aqueous phase to form a first product comprising one or more nucleotides; (e) removing the ethyl acetate from the oil phase to form a second products a oils with bioactives including tocopherols; (f) contacting the solid phase with a solvent under a condition and for a time sufficient to extract protein (i.e. zein) from the solid phase into the solvent, thereby forming a solid residual biomass phase and a liquid phase comprising the solvent and zein; (g) separating the solid residual biomass phase and the liquid phase comprising the solvent and zein; (h) remove the solvent from the liquid phase to give zein; and (i) drying the solid residue to give product as spent whole stillage.

In yet another aspect, the invention generally relates to a process for extracting one or more active ingredients from a biomass feedstock. The process includes: (a) contacting a biomass feedstock selected from a whole stillage with lipophilic solvent, preferable ethyl acetate; (b) separating lipid phase from the mixture; (c) removing solvents from the lipid phase to yield oils with bioactives including tocopherols and carotenoids; (d) contacting the remaining aqueous phase and solid mixture with alcohol to extract proteins (i.e., zein); (e) separating the liquid phase from the solid phase; (f) removing the solvent from the liquid phase to yield zein protein; (g) contacting the solid with water and nuclease to breakdown the RNA into nucleotides; (h) separating the solid residual biomass phase and the liquid phase comprising water and nucleotides; (i) remove the water from the liquid phase to give solid containing nucleotides; and (j) drying the solid residue to give product as spent whole stillage.

In yet another aspect, the invention generally relates to a composition comprising one or more active ingredients extracted by a process disclosed herein.

### BRIEF DESCRIPTION OF THE DRAWINGS

FIG. 1 schematically depicts an exemplary embodiment of the invention relating to extraction of whole stillage.

FIG. 2 schematically depicts an exemplary embodiment of the invention relating to extraction of whole stillage.

FIG. 3 depicts certain results from analysis of zein by SDS-PAGE.

FIG. 4 schematically depicts an exemplary embodiment of the invention relating to extraction of whole stillage.

FIG. 5 depicts certain results from an exemplary run. (A) Concentrations of GMP and AMP (mg/g dry weight) in whole stillage; (B) HPLC chromatogram of nuclease hydrolyzed whole stillage, peak at 17.397 min is for GMP and peak at 22.626 min is AMP.

FIG. 6 depicts certain results from an exemplary run. SDS-Page profile of zein. 1: Product 2 (zein extracted from whole stillage); 2: Zein extracted from DDGS; 3: Zein extracted from DDGS with 90% ethanol; 4: Zein from a commercial source.

FIG. 7 depicts certain results from an exemplary run. HPLC chromatogram of Product 3 revealing that it contains vanillic acid, caffeic acid, p-coumaric acid, and ferulic acid among a few other unknown compounds.

FIG. 8 depicts certain results from an exemplary run. HPLC chromatogram of Product 3, carotenoid fraction.

FIG. 9 depicts exemplary procedures of extraction of oils with bioactives and zein from corn bioethanol coproducts.

FIG. 10 depicts certain result of SDS-PAGE protein profile of zein.

FIG. 11 depicts certain results on yields of GMP and AMP yield (mg/g) from whole stillage hydrolysis (0.2% nuclease).

### DEFINITIONS

The term “beer”, as used herein, refers to the fermented corn biomass after it has been treated with starch hydrolases to break down the starch into fermentable sugar.

The term “bioavailability”, as used herein in the context of nutrition and nutritional ingredients, can be defined as the proportion of the administered substance capable of being absorbed and available for use or storage. Thus, bioavailability refers the fraction of a nutrient that is digested, absorbed and metabolized through normal pathways. (Srinivasan 2001 “Bioavailability of Nutrients: A Practical Approach to In Vitro Demonstration of the Availability of Nutrients in Multivitamin-Mineral Combination Products”. The Journal of Nutrition 131 (4 Suppl): 1349S-50S.)

The term “biomass”, as used herein, refers broadly to any biological material derived from living, or recently living organisms. Biomass can refer to plants or plant-based materials including woody biomass and agricultural biomass. Examples of biomass include corn syrup, molasses, silage, agricultural residues (corn stalks, grass, straw, grain hulls, bagasse, etc.), Whole Stillage, Thin Stillage, syrup, beer, Wet Distillers Grain with Solubles (WDGS), Modified WDGS, Distillers Dried Grains (DDG), Distillers Dried Solubles (DDS), Condensed Distillers Solubles (CDS), Distillers Dried Grains with Solubles (DDGS), kernel (corn kernel, etc.), syrup (corn syrups, etc.), woody materials (wood or bark, sawdust, timber slash, and mill scrap), poplars, willows, Eucalyptus, switchgrass, alfalfa, prairie bluestem, algae, including macroalgae, etc.). Examples of grain starch include: whole wheat flour, whole oats/oatmeal, whole grain corn/corn meal, brown rice, whole rye, whole grain barley, whole faro, wild rice, buckwheat, triticale, millet, quinoa, sorghum. Examples of starchy vegetables include: parsnip, plantain potato, pumpkin, acorn squash, butternut squash and green peas.

Exemplary biomass also include cellulosic material, lignocellulosic material, hemicellulosic material, carbohydrates, pectin, starch, inulin, fructans, glucans, corn, sugar cane molasses, sugar beet molasses, grasses, switchgrass, sorghum, high biomass sorghum, bamboo, algae and material derived from these. Biomass also includes processed or spent biomass, for example, after fermentation to produce alcohol or other fermentation products.

The terms “fermentation” or “fermenting”, as used herein, refer to the process of transforming an organic molecule into another molecule using a microorganism or group of microorganisms in or on a suitable medium for the microorganisms. The microorganisms can be growing aerobically or anaerobically. For example, “fermentation can refer to transforming sugars or other molecules from biomass to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid). Thus, fermentation includes alcohol fermentation.

Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms include, for example, yeast or filamentous fungi. The yeast can include strains from a Pichia or Saccharomyces species. In some embodiments, the yeast can be Saccharomyces cerevisiae. In some embodiments, the fermenting is effected by bacteria. In some embodiments, the microorganism (e.g., yeast or bacteria) can be a genetically modified microorganism.

The term “nuclease,” as used herein, refers to an agent, for example a protein or a small molecule, capable of cleaving a phosphodiester bond connecting nucleotide residues in a nucleic acid molecule. In some embodiments, a nuclease is a protein, e.g., an enzyme that can bind a nucleic acid molecule and cleave a phosphodiester bond connecting nucleotide residues within the nucleic acid molecule.

A nuclease may be an endonuclease, cleaving a phosphodiester bonds within a polynucleotide chain. Restriction endonucleases (restriction enzymes) cleave DNA at or near specific recognition sites (sequences). Non-specific endonucleases, on the other hand, cleave DNA or RNA into mono-, di-, tri-, or oligonucleotide products. Exemplary non-specific endonucleases include DNase I, S1 Nuclease, Nuclease Bal31 (which is also an exonuclease), Mung-bean nuclease, T7 Endonuclease I.

A nuclease may be an exonuclease, cleaving a phosphodiester bond at the end of the polynucleotide chain. These enzymes hydrolyze phosphodiester bonds from either the 3′ or 5′ terminus of polynucleotide molecules. Exonucleases that hydrolyze nucleotides from the 5′ end of a nucleic acid molecule may generally be referred to as a 5′→3′ exonucleases. Examples of 5′→3′ exonucleases include, by way of illustration only, lambda-exonuclease, T7 Exonuclease, and Rec J. Exonucleases that hydrolyze nucleotides from the 3′ end of a nucleic acid molecule may generally be referred to as a 5′→3′ exonucleases. Illustrative examples of 3′→5′ exonucleases include Exonuclease I, Exonuclease III, and Exonuclease T. Nuclease Bal31, which also functions as an endonuclease, is both a 5′→3′ exonuclease and an exonuclease. In some embodiments, exonucleases may be employed in which only functions exists, and other functions have been experimentally or naturally abolished. One non-limiting example of such a molecule is Klenow(5′→3′ exo) (which may also be written as Klenow(Asp³³⁵; Glu³⁵⁷; 5′→3′ exo.sup.−), in which the 5′→3′ exonuclease activity has been abolished, but the 3′→5′ exonuclease activity of the enzyme is retained. It will be readily appreciated by a skilled artisan that additional endonucleases and exonucleases exist which, though not listed above, are suitable for use within the context of the present invention, without departing from the sprint and scope thereof.

In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease recognizes a single stranded target site, while in other embodiments, a nuclease recognizes a double-stranded target site, for example a double-stranded DNA target site. The target sites of many naturally occurring nucleases, for example, many naturally occurring DNA restriction nucleases, are well known to those of skill in the art.

Exemplary nucleases include exonuclease and endonuclease. The Exonuclease further include Exodeoxyribonucleases and oligonucleotidase. One example is EC 3.1.13.3, oligoribonuclease, which is an exoribonuclease derived from Flammulina velutipes. Endonuclease further include endodeoxyribonuclease and endoribonuclease. endodeoxyribonuclease further includes deoxyribonuclease I, deoxyribonuclease II, .deoxyribonuclease IV, restriction enzyme, UvrABC endonuclease. Deoxyribonuclease IV further includes endodeoxyribonuclease IV, E. coli endonuclease IV, endodeoxyribonuclease, redoxyendonuclease, deoxriboendonuclease, Escherichia coli endonuclease II, endonuclease II, which catalyzes endonucleolytic cleavage to 5′-phosphooligonucleotide end-products. Endorebonuclease further includes RNase III, RNase H, RNase P, RNase A, RNase T1. The nuclease further includes phosphodiesterase. Phosphodiesterase further include autotaxin Phospholipase, Sphingomyelin phosphodiesterase, PDE1, PDE2, PDE3, PDE4A/PDE4B, PDE5, Lecithinase (Clostridium perfringens alpha toxin), cyclic nucleotide phosphodiesterase. Phosphodisterase further include those obtained or isolated from microbes including yeasts, bacteria fungus. Preferably the phosphodiesterase is from ascomycetous fungi. Further preferably, the phosphodiesterase is from penicillium genus of ascomycetous fungi. phosphodiesterase is obtained from penicillium Penicillium bilaiae, Penicillium camemberti, Penicillium candida, Penicillium chrysogenum, Penicillium citrinum, Penicillium claviforme, Penicillium crustosum, Penicillium dititatum, Penicillium divaricatum, Penicillium expansum, Penicillium glaucum, Penicillium granulatum, Penicillium marneffei, Penicillium notatum, Penicillium purpurogenum Penicillium roqueforti, Penicillium spiculisporum, Penicillium stoloniferum, Penicillium varians, Penicillium viridicatum, Penicillium verrucosum, Penicillium commune.

The term “nucleic acid”, as used herein, refers to a polymer of any length, e.g., greater than about 2 bases, greater than about 10 bases, greater than about 100 bases, greater than about 500 bases, greater than 1,000 bases or more bases composed of nucleotides, e.g., deoxyribonucleotides or ribonucleotides. A nucleic acid may exist in a single stranded or a double-stranded form. A double stranded nucleic acid has two complementary strands of nucleic acid may be referred to herein as the “first” and “second” strands or some other arbitrary designation.

The term “nucleotide”, as used herein, refer to nucleoside monophosphate, a sub-unit of a nucleic acid (whether DNA or RNA or analogue thereof), which includes a phosphate group, a sugar group and a heterocyclic base, as well as analogs of such sub-units.

The terms “pre-treatment” or “pre-treating”, as used herein, refer to any mechanical, thermal, biochemical or chemical process, or combination thereof, that render the biomass more susceptible to extraction with a solvent such as alcohol or aqueous alkaline solution.

The term “stillage”, as used herein, refers to the mixture of non-fermentable (or non-fermented) dissolved solids, insoluble grain fines proteins, dead yeasts and water, which are the residues after removal of ethanol from a fermented beer by distillation. Stillage may be dried to recover the solids material (as DDG in the case of feedstocks).

The term “thin stillage”, as used herein, refers to the liquid portion of stillage separated from the solids by screening or centrifuging. This stillage contains some yeast, suspended fine particles and dissolved material. This stillage is normally sent to an evaporator to be concentrated to a thick syrup and then dried with the solids portion to give DDGS.

The term “whole stillage”, as used herein, refers to the entire stillage emerging from a distillation unit before any removal of solids by screening or centrifuging.

### DETAILED DESCRIPTION OF THE INVENTION

The invention provides novel and improved methods that allow effective capture of valuable active ingredients in whole stillage and thin stillage at cost-effective commercial scale. Active ingredients that can be efficiently captured include, for example, vitamins, flavonoids, carotenoids, tocopherols, and lipophilic phenolics, phenolic acids and nucleotides. The invention also provides novel compositions and formulations of active ingredients with unique properties (e.g., higher nutritional values and enhanced bioavailability). Additionally, the invention alleviates environmental burden and reduces cost of biofuel production through efficient and cost-effective utilization of biomass.

Efficient and cost-effective recovery of active ingredients from whole stillage or thin stillage at industrial scales presents a number of challenges. Typical difficulties include extraction of specific ingredients that can meet the market demand, low overall extraction yields, inferior quality of extracts comparing to competitive products manufactured from other raw materials, such as corn per se. Thus, a well-designed process, carefully selected processing conditions, as well as precise operating protocols are needed to balance the different factors and optimize the overall efficiency and productivity.

Processes of the invention may be employed to extract active ingredients from various biomass feedstock, for example, selected from whole stillage, thin stillage, Distillers Dried Grains with Solubles (DDGS), syrup, beer, Wet Distillers Grain with Solubles (WDGS), and Modified Wet Distillers Grain with Solubles (Modified WDGS). In certain preferred embodiments, the biomass feedstock comprises a whole stillage of grains selected from corn, rice, wheat, barley and rye. In certain preferred embodiments, the biomass feedstock comprises a thin stillage of grains selected from corn, rice, wheat, barley and rye. In certain preferred embodiments, the biomass feedstock comprises a Distillers Dried Grains with Solubles (DDGS) of grains selected from corn, rice, wheat, barley and rye. In certain preferred embodiments, the biomass feedstock comprises a Wet Distillers Grain with Solubles (WDGS) of grains selected from corn, rice, wheat, barley and rye. In certain preferred embodiments, the biomass feedstock comprises a Modified Wet Distillers Grain with Solubles (Modified WDGS) of grains selected from corn, rice, wheat, barley and rye. In certain preferred embodiments, the biomass feedstock comprises a beer of grains selected from corn, rice, wheat, barley and rye.

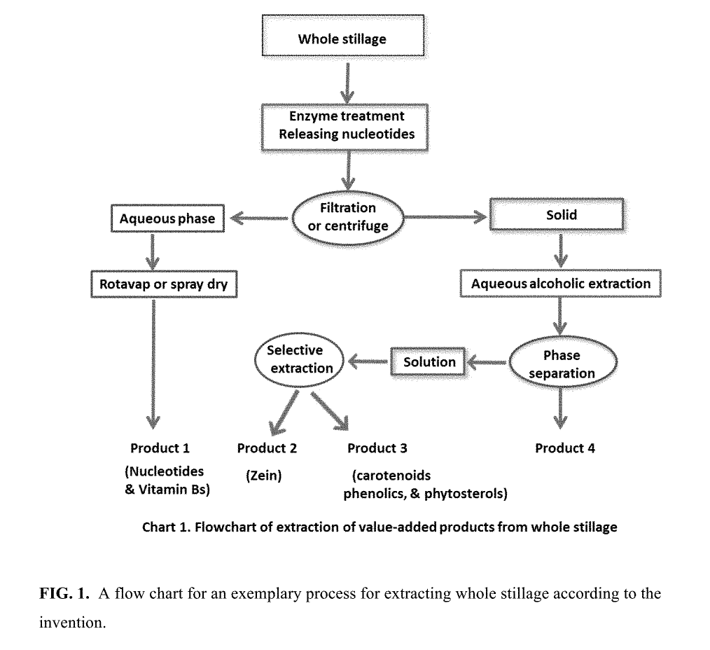

Referring to FIG. 1, which depicts a flow chart (100) describing an exemplary embodiment of the invention for extracting active ingredients from whole stillage or thin stillage. Whole stillage feedstock is treated with an enzyme to release nucleotides. The mixture then is separated into an aqueous phase and a solid phase by filtration or centrifuge. The aqueous phase is rotovaped or spray dried to afford a mixture of nucleotides and vitamins (Product 1). Product 1 are further separated into nucleotide components and vitamin components.

An aqueous alcoholic extraction is carried out on the solid phase. A phase separation follows yielding a solid product (Product 4) and an aqueous material, which is further extracted to yield zein (Product 2) and a mixture of carotenoids, phenolics and phytosterols (Product 3). Details of the process are provided in the examples section herein.

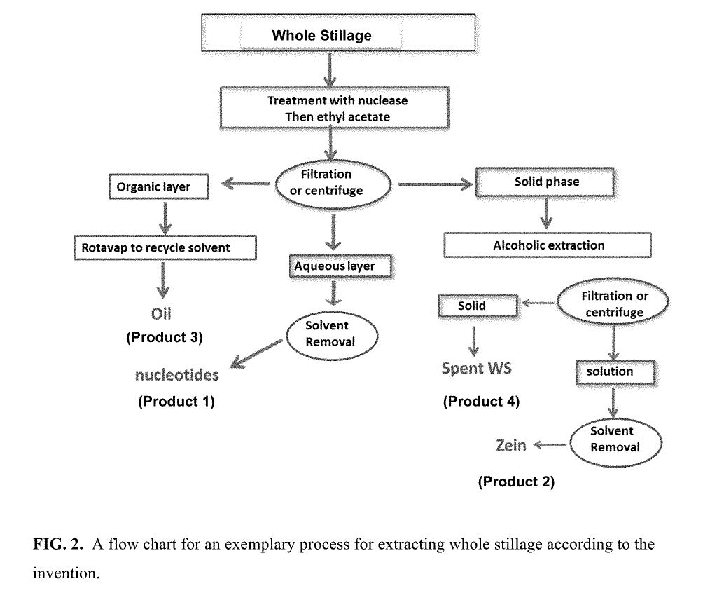

Referring to FIG. 2, which depicts a flow chart describing another exemplary embodiment of the invention for extracting active ingredients from whole stillage or thin stillage.

In one aspect, the invention generally relates to a process for extracting one or more active ingredients from a biomass feedstock. The process includes: (2) contacting a biomass feedstock (e.g., selected from a whole stillage or thin stillage) with an enzyme capable of causing the biomass feedstock to release one or more nucleotides, thereby forming a mixture of a solid phase and an aqueous phase comprising one or more nucleotides; (b) separating the solid phase from the aqueous phase; (c) removing water from the aqueous phase to form a first product comprising one or more nucleotides; (d) contacting the solid phase with a solvent under a condition and for a time sufficient to extract the one or more active ingredients from the solid phase into the solvent, thereby forming a solid residual biomass phase and a liquid phase comprising the solvent and the extracted one or more active ingredients; (e) separating the solid residual biomass phase and the liquid phase comprising the solvent and one or more active ingredients; (f) selectively extract the liquid phase to yield a second product; (g) selectively extract the liquid phase to yield a third product; and (h) selectively extract the solid residual biomass phase to yield a fourth product.

Any suitable whole stillage or thin stillage feedstock may be used. Exemplary feedstock includes fermentation products of plants or plant-based materials. In certain embodiments, the biomass feedstock is fermentation products of various grains (e.g., corn, rice, wheat, barley and rye). In certain preferred embodiments, the feedstock is whole stillage. In certain preferred embodiments, the feedstock is whole stillage produced from corn-based ethanol fermentation.

In certain embodiments, the biomass feedstock comprises a whole stillage of grains selected from corn, rice, wheat, barley and rye. In certain embodiments, the biomass feedstock comprises a thin stillage of grains selected from corn, rice, wheat, barley and rye.

In certain embodiments, the enzyme is a nuclease.

In certain embodiments, contacting the biomass feedstock with the enzyme is performed at a temperature between about 15° C. to about 70° C. (e.g., between about 20° C. to about 70° C., between about 25° C. to about 70° C., between about 30° C. to about 70° C., between about 35° C. to about 70° C., between about 40° C. to about 70° C., between about 20° C. to about 60° C., between about 25° C. to about 60° C., between about 30° C. to about 60° C. between about 40° C. to about 60° C., between about 15° C. to about 55° C., between about 15° C. to about 50° C., between about 15° C. to about 45° C., between about 15° C. to about 40° C., between about 15° C. to about 35° C.) for a time from about 0.5 hour to about 30 hours (e.g., from about 1 hour to about 30 hours, from about 1 hour to about 24 hours, from about 2 hours to about 20 hours, from about 3 hours to about 20 hours, from about 5 hours to about 20 hours, from about 8 hours to about 20 hours, from about 10 hours to about 20 hours, from about 0.5 hour to about 10 hours, from about 0.5 hour to about 8 hours, from about 0.5 hour to about 5 hours, from about 0.5 hour to about 3 hours, from about 1 hour to about 10 hours, from about 1 hour to about 5 hours, from about 1 hour to about 3 hours).

In certain embodiments, the first product comprises one or more nucleotides and one or more vitamins. The nucleic acids recovered in the extract may include RNA molecules, DNA molecules or both, for example, yeast RNA and yeast DNA. The exaction may yield a mixture of 5′-nucleotide monophosphate (MP) monomers selected from GMP, UMP, AMP, and CMP. In certain embodiments, the process further includes separating the GMP, UMP, IMP and CMP into substantially pure forms. The separation and/or isolation of GMP, UMP, IMP and CMP may be carried out by any suitable techniques, for example, by anion exchange chromatography.

In certain embodiments, the one or more nucleotides comprise AMP and GMP.

In certain embodiments, the one or more vitamins comprise one or more of vitamin E, vitamin B (e.g., vitamin B1, B2, B3, B4, B6), vitamin D, vitamin A.

In certain embodiments, the second product comprises zein (e.g., α-zein).

In certain embodiments, the third product comprises one or more of lutein, vanillic acid, caffeic acid, p-coumaric acid and ferulic acid.

In certain embodiments, the fourth product comprises one or more of fibers and proteins.

Any suitable solvent (e.g., alcohol, water, acetone, ethyl acetate, and hexanes, including solvent mixtures thereof) may be used for extraction. In certain embodiments, an alcohol is employed as the solvent. In certain preferred embodiments, ethanol is employed as the solvent.

In certain embodiments, the solvent may include two or more co-solvents, for example, a first or primary co-solvent and a second or secondary co-solvent. In certain embodiments, ethanol is employed as the primary co-solvent and a co-solvent is also used. The weight ratio of the first, primary co-solvent to the second, secondary co-solvent may be any suitable ratio, for example, from about 0.01:1 to about 100:1 (e.g., from about 0.1:1 to about 100:1, from about 0.5:1 to about 100:1, from about 1:1 to about 100:1, from about 3:1 to about 100:1, from about 5:1 to about 100:1, from about 10:1 to about 100:1, from about 0.01:1 to about 80:1, from about 0.01:1 to about 50:1, from about 0.01:1 to about 20:1, from about 0.01:1 to about 10:1, from about 0.01:1 to about 5:1, from about 0.01:1 to about 3:1). In certain preferred embodiments, the first co-solvent is ethanol.

Separation of a solid phase and a liquid phase may be carried out by any suitable methods, for example, by filtration and centrifuge.

A solvent may be removed by a variety of techniques, for example, by evaporation, distillation, vacuum transfer, and filtration. Evaporation can be conducted under a raised temperature and/or a reduced pressure. Temperatures and pressures suitable for solvent removal may be selected dependent on the nature of the solvent, the scale of production, whether the recovered solvent is to be recycled and reused in extraction, etc. Generally, evaporation may be effectively carried out at a temperature from about 20° C. to about 100° C. (e.g., from about 30° C. to about 100° C., from about 40° C. to about 100° C., from about 50° C. to about 100° C., from about 60° C. to about 100° C., from about 20° C. to about 100° C., from about 20° C. to about 100° C., from about 20° C. to about 100° C., from about 20° C. to about 100° C.) and at a pressure from about atmospheric pressure to about 1 mmHg. In certain embodiments, the removed solvent from the liquid phase is recycled and used in the extraction step.

It is noted that while the process may be generally performed as a batch process at different scales (e.g., at least 5 Kg of feedstock per batch, at least 10 Kg of feedstock per batch, at least 20 Kg of feedstock per batch, at least 200 Kg of feedstock per batch, at least 1,000 Kg of feedstock per batch), the process may be designed as a continuous process whereby biomass feedstock is replenished continuously or periodically with a continuous extraction, residual separation and/or solvent removal.

In another aspect, the invention generally relates to a process for extracting one or more active ingredients from a biomass feedstock. The process includes: (a) contacting a biomass feedstock selected from a whole stillage with an enzyme capable of causing the biomass feedstock to release one or more nucleotides, thereby forming a mixture of a solid phase and an aqueous phase comprising one or more nucleotides; (b) adding lipophilic solvent, preferable ethyl acetate to extract the lipid soluble components from the mixture, (c) separating oil phase, water phase, and solid phase; (d) removing water from the aqueous phase to form a first product comprising one or more nucleotides; (e) removing the ethyl acetate from the oil phase to form a second products a oils with bioactives including tocopherols; (f) contacting the solid phase with a solvent under a condition and for a time sufficient to extract protein (i.e., zein) from the solid phase into the solvent, thereby forming a solid residual biomass phase and a liquid phase comprising the solvent and zein; (g) separating the solid residual biomass phase and the liquid phase comprising the solvent and zein; (h) remove the solvent from the liquid phase to give zein; and (i) drying the solid residue to give product as spent whole stillage.

In yet another aspect, the invention generally relates to a process for extracting one or more active ingredients from a biomass feedstock. The process includes: (a) contacting a biomass feedstock selected from a whole stillage with lipophilic solvent, preferable ethyl acetate; (b) separating lipid phase from the mixture; (c) removing solvents from the lipid phase to yield oils with bioactives including tocopherols and carotenoids; (d) contacting the remaining aqueous phase and solid mixture with alcohol to extract proteins (i.e., zein); (e) separating the liquid phase from the solid phase; (f) removing the solvent from the liquid phase to yield zein protein; (g) contacting the solid with water and nuclease to breakdown the RNA into nucleotides; (h) separating the solid residual biomass phase and the liquid phase comprising water and nucleotides; (i) remove the water from the liquid phase to give solid containing nucleotides; and (j) drying the solid residue to give product as spent whole stillage.

In yet another aspect, the invention generally relates to a composition comprising one or more active ingredients extracted by a process disclosed herein.

Depending on the source of the feedstock, a variety of compounds can be obtained. Preferably, the one or more active ingredients are selected from vitamins, flavonoids, carotenoids, tocopherols, and lipophilic phenolics, and phenolic acids. In certain embodiments, the flavonoids that may be extracted by the processes herein include one or more of: anthocyanins (including both sugar-free anthocyanidin aglycones and anthocyanin glycosides). In certain embodiments, the carotenoids that may be extracted by the processes herein include one or more of: beta-carotene, lutein, and zeaxanthin. In certain embodiments, the tocopherols that may be extracted by the processes herein include one or more of: alpha-tocopherol, delta-tocpherol, and gamma-tocopherol.

Depending on the source of feedstock and particular extraction conditions, the relative yields of active ingredients may vary, which can be utilized to control the compositions of the resulting extract. For instance, corn-based whole or thin stillage usually are higher in carotenoids than wheat-based corn-based whole or thin stillage. Actual yield of recovery of a particular ingredient depends on factors such as source of feedstock, solvent choice, ratio to biomass, temperature and length of extraction, etc. The process can be designed to be suitable for extracting one or more specific active ingredients or class(s) of compounds.

In certain embodiments, the recovery yield for nucleotides is 1% or greater, for example from about 20% to 95%, preferably from about 50% to 95%, more preferably from about 70% to about 95%, and most preferably about 90% to about 100%.

In certain embodiments, the recovery yield for vitamins is 1% or greater, for example from about 20% to 95%, preferably from about 50% to 95%, more preferably from about 70% to about 95%, and most preferably about 90% to about 100%.

In certain embodiments, the recovery yield for carotenoids, is 1% or greater, for example from about 20% to 95%, preferably from about 50% to 95%, more preferably from about 70% to about 95%, and most preferably about 90% to about 100%.

In certain embodiments, the recovery yield for lipophilic phenolics, is 1% or greater, for example from about 20% to 95%, preferably from about 50% to 95%, more preferably from about 70% to about 95%, and most preferably about 90% to about 100%.

The process of the invention may include a pre-treatment step, for example, to prepare the biomass feedstock to be more suitable for a particular extraction and/or separation techniques. For instance, the feedstock (e.g., whole stillage) may be concentrated to a level suitable for efficient and effective extraction as well as separation with filtration and/or centrifugation. Other pre-treatment techniques include, for example, membrane separation, freeze and decantation to separate lipids from the water phase.

It is noted that for certain applications, it may be beneficial to conduct a repeat (e.g., a second or a third) round of extraction, separation and solvent removal to an extract product of the desired compositions. The repeat round may be identical to the previous round. The repeat round may also be different from the previous round in one or more aspects, for example, solvent choice and amount, length of extraction, techniques of residual biomass separation and removal of solvent.

Depending on the source of biomass, extraction conditions (e.g., solvent choice, solvent to biomass ratio, extraction temperature and length of time), method of separation and solvent removal, the compositions of the feedstock extract may be varied. Thus, the extraction can be processed such as to result in certain compositions of active ingredients.

### EXAMPLES

### Example I. Extraction of Active Ingredients from Whole Stillage Procedure One

FIG. 1 depicts a flow chart for an exemplary process for extraction of active ingredients according to the invention. This process allows fractionation of the whole stillage into various products

Extraction of Yeast Nucleotides and Vitamins (Product 1) from Whole Stillage

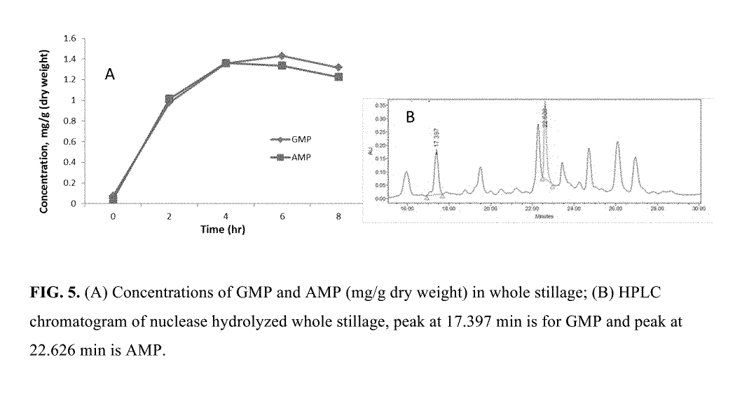

Whole stillage (14.33 g, water content 87%, dry weight, 1.8 g, sample obtained from Three Rivers Energy LLC, Coshocton, Ohio, USA) was mixed with water (6 mL) and nuclease (0.2%) and stirred at temperature between 20 to 50° C. for a few hours. The adenosine monophosphate (AMP) and guanine monophosphate (GMP) contents are monitored by HPLC and the results are shown in FIG. 5. The resulting slurry was filtered and the solution is evaporated to dryness to give Product 1, weight: 0.294 g (Yield: 16.2%). HPLC analysis shows that it contains 3.9 mg/g AMP and GMP respectively.

### Extraction of Zein (Product 2) and Bioactives (Product 3)

The residue from the above [0068] step was mixed with 70% ethanol (weight to volume ratio ranging from 1:5 to 1:20) and heated for one hour at 60° C. oil bath. The slurry was filtered and the residue was extracted with 95% ethanol twice (equal volume) in the same fashion. The residue is Product 4, which is mainly fibers and proteins. The filtrates were combined and the volatiles were evaporated to give oily residue. Anhydrous ethanol was added and stirred at room temperature for 10 minutes. The mixture was centrifuged and the supernatant was decanted to another flask. The residue was washed two more times with ethanol. The ethanol solution was combined. The residue was then dried in vacuum to give solid, which is Product 2 (zein). Yield, 0.134 g (7.4%). The ethanol solution was evaporated to give Product 3 as oil. Yield: 0.21 g (11.6%).

In total, the extraction yields of whole stillage based on dry weight is 35.2%.

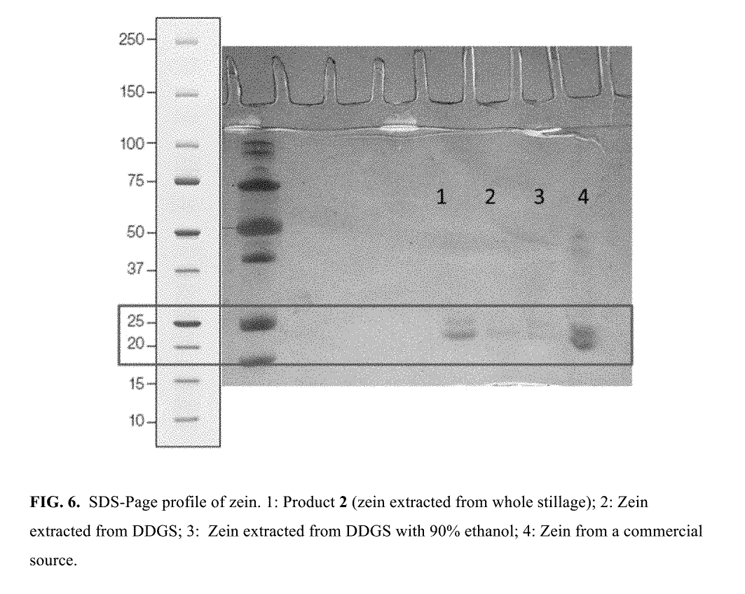

Sodium dodecyl sulfate-polyacrylamide gel electrophoresis (SDS-PAGE) analysis of Product 2 shown that it contains α-zein (lane 4) as the sole protein (FIG. 6). The results also demonstrated that whole stillage contains mainly α-zein (19 and 22 kDa). From this result, the whole stillage is a good material for extraction of α-zein.

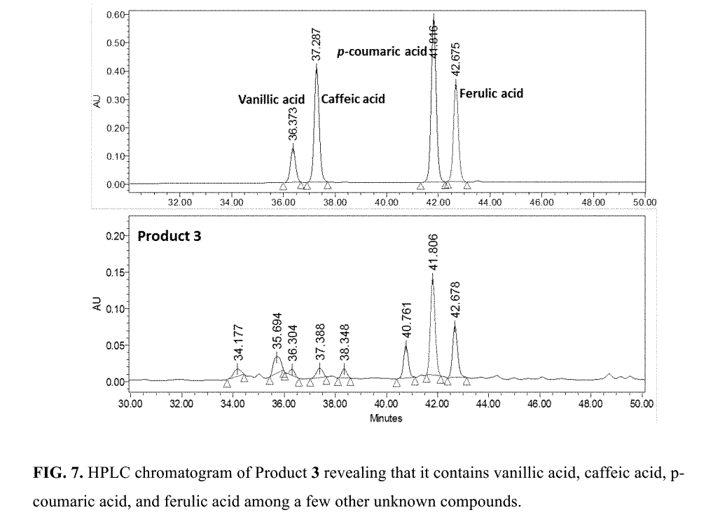

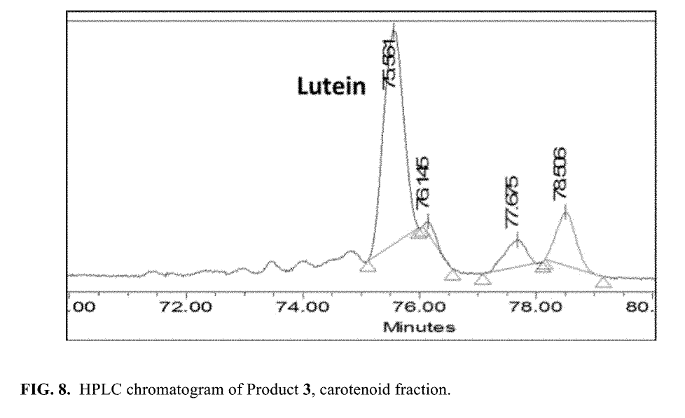

HPLC analysis of Product 3 shown that it contains vanillic acid, caffeic acid, p-coumaric acid, and ferulic acid as well as a few uncharacterized compounds that are likely phenolic acids (FIG. 7). In addition, it contains significant amount of lutein (FIG. 8). The concentrations of the compounds in Product 3 are: Lutein: 0.63 mg/g; vanillic acid, 0.055 mg/g; p-coumaric acid, 0.89 mg/g; ferulic acid, 0.051 mg/g.

### Example II. Extraction of Active Ingredients from Whole Stillage by Procedure Two

FIG. 2 depicts a flow chart for an exemplary process for extraction of active ingredients according to the invention. This process allows fractionation of the whole stillage into various products.

Extraction of Yeast Nucleotides (Product 1) from Whole Stillage

Whole stillage was received from Plymouth Energy, LLC, Iowa, USA. It contains moisture of 86%. Vitamin B contents of the whole stillage include: vitamin B2, 0.11 mg/100 g (wet weight); vitamin B3, 5.45 mg/100 g (wet weight); vitamin B9, 2.97 μg/100 g (wet weight), choline, 19 mg/100 g (wet weight), vitamin B5, 43.1 μg/100 g (wet weight).

The whole stillage (5 KG) was added to a double-layer glass reactor followed by addition of 5′-phosphodiesterase (50 g). The reactor was heated to reach 60° C. and held for 24 hours. Ethyl acetate (5 L) was added to the reactor. The temperature of the reactor was cooled to 30° C. The mixture was decanted to a container. The slurry was centrifuged at 3000 rpm for 15 minutes to separate residue from the filtrate, which was collected. Filtrate was separated. The water phase was added to the reaction kettle, and the organic phase was collected. Ethyl acetate (5 L) was added to the residue in the reactor, which was then heated to reach 60° C. and held for 1 hour. The temperature of the reactor was then cooled to 30° C. This mixture was decanted to a container. The slurry was centrifuged at 3000 rpm for 15 minutes to separate residue from the filtrate, which was collected. The filtrate was separated. The water phase was spray dried with temperature of the spray head set at 220° C. and flow rate of the liquid set at 18 ml/min. The solid obtained was the crude nucleotide fraction as tan soli as PRODUCT 1 245 g (32.7% based on dry weight). The product contents moisture 7.5%, protein 25.1%, and ash 16.4%.

The organic phase was concentrated by a rotary evaporator fitted with a cooling circulator to recycle the solvents. Temperature of the rotary evaporator was set at 45° C. to 50° C. After the oil volatiles were removed, PRODUCT 3 is obtained, yield: 126.6 g (16.9% based on dry weight).

### Extraction of Zein (Product 2)

Ethanol solution (5 L, 70%) was added to a double-layer glass reactor and the speed of the stirrer was set to 280 rpm. The last residue from above procedure was added to the reactor. The reactor was then heated to reach 60° C. and held for 1 hour. The temperature of the reactor was then cooled to 30° C. The slurry was collected to two 5 L-plastic beakers. Centrifuge at 3000 rpm for 3 minutes was used to separate the residue and the filtrate, which was stored in a 5 L plastic beaker.

Ethanol solution (5 L, 70%) was added to the double-layer glass reactor. The speed of the stirrer was set to 280 rpm and the last residue was added to the reactor. The reactor was then heated to reach 60° C. and held for 1 hour. The temperature of the reactor was then cooled to 30° C. The slurry was collected to two 5 L-plastic beakers. Centrifuge at 3000 rpm for 3 minutes was used to separate the residue and the filtrate, which was stored in a 5 L plastic beaker.

The filtrate so obtained was concentrated using a rotary evaporator fitted with a cooling circulator to recycle the solvents. The rotary evaporator was set at 50° C.-60° C. and the vacuum was set at 13.4 PSI-13.8 PSI.

Semi solid (83.9 g) was obtained with a solvent recovery of 7.2 L. The semi solids were dried at 65° C. under vacuum for 15 hours at −14.4 PSI to obtain an amorphous solid as zein fraction. The total zein fraction (55.7 g) represented an extraction yield of 7.4%. The product contains moisture (4.2%), ash (8.3%), and total protein (31.6%).

### Purification of Zein

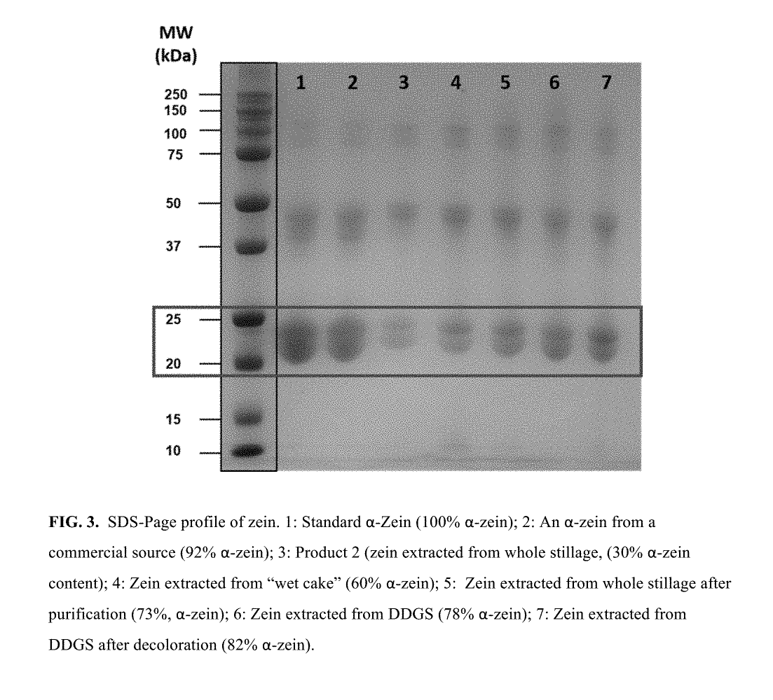

Zein (10 g) extracted from whole stillage as described in 0080, was mixed with 100 mL deionized water, (100 mL), ethyl acetate (100 mL) in a round-bottom flask (250 mL). The mixture was allowed to heat to 55° C. with stirring for 20 min. After cooling down, the mixture was decanted to a centrifuge bottle and centrifuged at 3000 rpm for 15 minutes to separate residue from the filtrate. Then, the residue was mixed with another 100 mL deionized water and 100 mL ethyl acetate, and the same procedure was repeated once more. The residue was then dried in vacuum to obtain light yellow zein powder (2 g, 20%). The SDS-PAGE analysis of the zein is shown in FIG. 3 (lane 5). The purity of α-zein was estimated from FIG. 3.

### Spent Whole Stillage (Product 4)

The residue was dried in a drying oven at 100° C. at 1 atm for 24 hours to obtain spent whole stillage (320.9 g) representing a yield of 42.9%. The product contains moisture (4.6%), ash (1.03%), and total protein (34.1%).

### Example III. Extraction Whole Stillage by Procedure Three

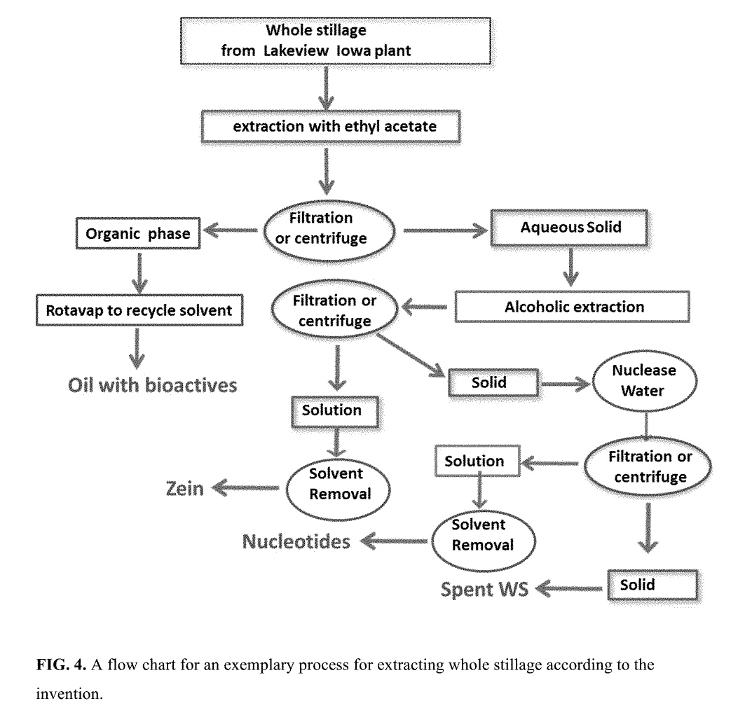

FIG. 4 depicts a flow chart for an exemplary process for extraction of active ingredients according to the invention.

In a 250 single-neck flask, whole stillage (50 g, sample from Plymouth Energy LLC, IA, USA) was placed and mixed with ethyl acetate (50 mL), the mixture was heated at 60° C. for 30 min with constant stirring. After the stirring stopped and the solid settled to the bottom of the flask, the liquid phase was decanted and filtered. The organic layer was separated using as separation funnel while the aqueous layer was combined with the solid residue. The combined slurry was extracted two more times with ethyl acetate (60° C. 30 min). After the organic phase was separated. The volatiles were evaporated by a rotary evaporator to yield yellow oil fraction 1.1836 g (14.4%, based on dry weight). Analytical results for the oils are: total phytosterols content: 7.4 mg/g, and the total carotenoid content is 0.52 mg/g.

The combined aqueous and solid residue was mixed with anhydrous ethanol (150 mL) and heated with stirring at 60° C. for 30 min. The mixture was centrifuged and the supernatant was separate from the solid residue. The solid residue was extracted two more times with ethanol (60° C., 30 min) and the ethanolic solutions were combined and evaporated by rotary evaporator to give zein fraction 2.1716 g (26.4% based on dry weight).

The residue was mixed with water (50 mL) and nuclease (0.5 g) and heated at 60° C. for 17 h. The resulting slurry was centrifuged. The supernatant was decanted and the volatiles were removed by rotary evaporator to give nucleotide fraction 0.9733 g (yield: 11.8%, based on dry weight). Analytical results for this fraction include: CMP, 0.01%; UMP, 0.018%, GMP, 0.01%; AMP, 0.02%. Reducing sugar 10.9%, total carbohydrate: 18.9%.

The solid residue from the above process was dried to give spent whole stillage 4.2573 g (51.9% based on dry weight).

### Example IV. Extraction of Oil Soluble Active Ingredients and Zein from Whole Stillage, Beer, Syrup; WDGS, and DDGS

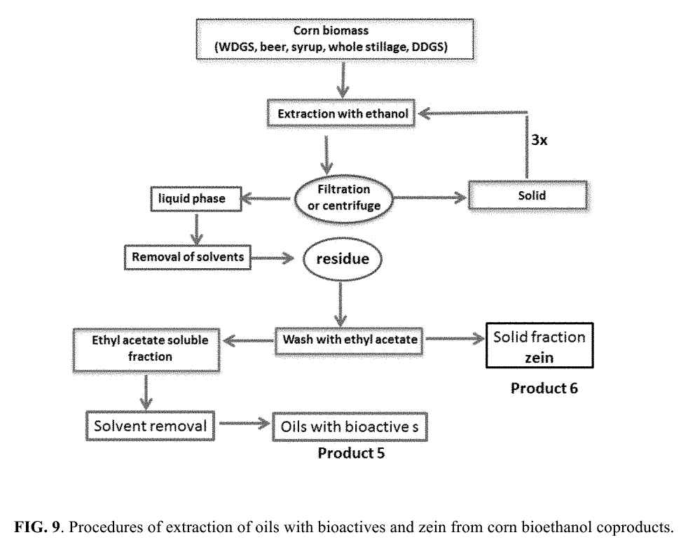

FIG. 9 depicts a flow chart for an exemplary process for extraction of active ingredients according to the invention. This process allows fractionation of the biomass into various products. Samples were obtained from Three Rivers Energy LLC, Coshocton, Ohio, USA

Two grams of each sample were extracted three times successively with ethyl acetate (100 mL), absolute ethanol (100 mL), and 70% ethanol (100 mL). Each extraction was operated by shaking 5 h and followed by centrifugation and filtration to obtain the solutions. The solutions were combined and the solvent was removed by rotary evaporator under reduced pressure. The recovered oil mixture was washed by ethyl acetate (EA, 10 mL) for three times to separate other lipids from zein. The EA-insoluble residues (mainly zein), were subsequently dissolved in 70% ethanol (30 mL). The solvents were removed under reduced pressure again to afford oil fraction and zein fraction respectively. The processes were repeated three times to obtain standard deviations for the yields of each fraction. The yields of oil fraction and zein fraction are stated in Table 1.

TABLE 1
Yields (wt % on dried matter) of oil
and zein from bio-ethanol co-products

| | Beer | Whole stillage | Syrup | WDGS | DDGS |
|---|---|---|---|---|---|
| Oil | 12.5 | 12.3 | 3.2 | 7.6 | 7.2 |
| Zein | 5.2 | 6.1 | N.A. | 6.5 | 6.78 |

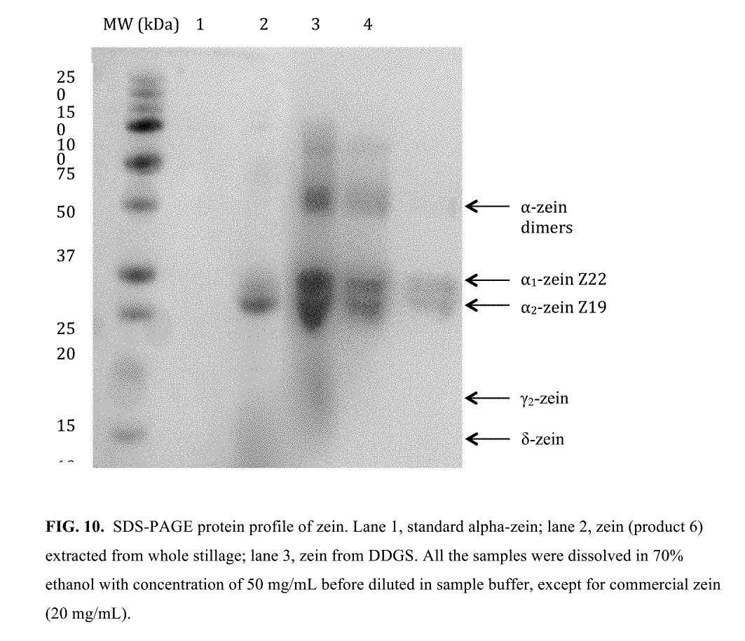

The oil was dissolved in ethanol with concentration of 50 mg/mL for HPLC analysis. The zein was dissolved in aqueous ethanol with concentration of 50 mg/mL for SDS-PAGE analysis and the results are shown in FIG. 10. The phenolic acid contents of the oil fractions are shown in Table 2.

TABLE 2
Yield of lipophilic compounds in oil fractions extracted from the biomass (mg/100 g oil)

| | Vanillic | Caffeic | p-Coumaric | Ferulic | | |
|---|---|---|---|---|---|---|
| | acid | acid | acid | acid | Lutein | α-Tocopherol |
| Retention time (min) | 36.37 | 37.29 | 41.82 | 42.68 | 75.56 | 84.74 |
| Beer | — | 7.08 | 1.91 | 2.52 | 62.95 | 11.68 |
| Whole stillage | 5.49 ± 0.36 | — | 8.61 ± 0.28 | 5.11 ± 0.17 | 62.97 ± 3.34 | 52.80 ± 2.26 |
| Syrup | — | 6.34 | 6.41 | 6.79 | 24.98 | 11.68 |
| WDGS | — | 5.72 | 13.21 | 12.25 | 76.37 | 54.42 |
| DDGS | 8.74 ± 0.52 | 8.68 ± 0.47 | 14.49 ± 1.96 | 16.38 ± 0.87 | 31.62 ± 2.03 | 40.12 ± 1.23 |

The zein fraction (product 6, cf. FIG. 9) extracted from whole stillage (from Three River Energy LLC, Ohio, USA) and that of DDGS were analyzed by SDS-PAGE electrophoresis and the results are depicted in FIG. 10. The quality of the zein extracted from whole stillage and from DDGS was further analyzed according to their contents of α-zein by UV-VIS absorbance at 650 nm of the suspension of the zein in water and the results are shown in Table 3. The quality of the zein is referenced to that of standard α-zein. The zein from whole stillage has much higher quality in comparison with that of DDGS.

TABLE 3
UV-vis absorbance (650 nm) of α-zein bands in SDS-PAGE

| | Concentration | | Equivalent of |
|---|---|---|---|
| | (mg/mL) | *Abs | commercial zein |
| Zein from Whole stillage | 50 | 0.284 | 1.378 |
| Zein from DDGS | 50 | 0.165 | 0.752 |

Standard curve (with commercial zein): Abs = 0.0038Con. + 0.0221 (R² = 0.9899)
*The total absorbance of α₁-zein Z22 α₂-zein Z19 bands in SDS-PAGE was measured with microplate reader (Bio-Tek, USA).

### Example V. Extraction of RNA from DDGS, WDGS, Whole Stillage and Beer

DDGS (100 g) was mixed with water (300 mL) and sodium chloride (24 g) and heated at 90° C. for four hours then cooled to 4° C. before the solution was filtered and the filtrate was acidified to pH 2.5 and kept in 4° C. overnight. The precipitate was filtered and the residue was washed with absolute ethanol and dried to give RNA extracts. The RNA in WDGS, Whole stillage and “beer” were extracted with the same process and the estimated RNA contents were measured by UV-VIS spectrometer. The concentrations of RNA of the samples are shown in Table 4.

TABLE 4
Nucleic acid contents of distiller biomass on dry weight basis

| Sample | Extraction yields* (‰) |
|---|---|
| Yeast | 26.7 |
| DDGS | 0.151 |
| WDGS | 0.112 |
| Whole stillage | 0.126 |
| Beer | 0.118 |

*Calculated based on the absorbance value at 260 nm (One absorbance unit equals to 40 μg/mL)

### Example VI. Extraction of Nucleotides

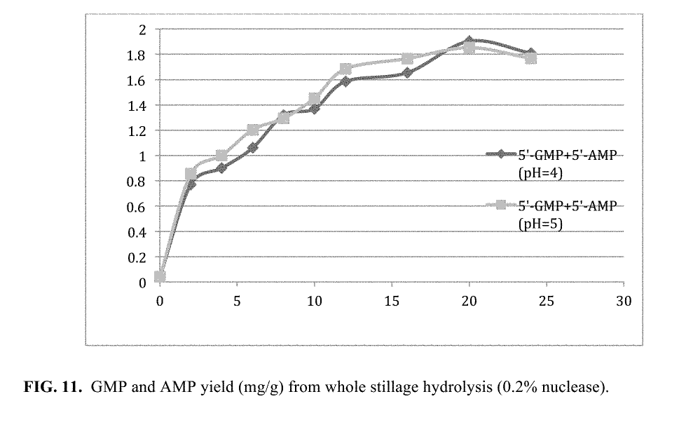

A water solution of the whole stillage at 10% on dry weight was hydrolyzed by 0.2% of Nuclease P1 at pH 4.0 and 60° C. The progress of the hydrolysis was monitored by HPLC by taking samples at specific time points. The sample was then centrifuged to separate the solution and solids. The solution was filter by 0.45 μm micro-filter and analyzed by HPLC to determine the nucleotide concentrations depicted in FIG. 11. To evaluate the impact of pH, another hydrolysis was carried out at pH 5.0.

### Example VII. HPLC Quantification Method of Bioactives in the Oil Fractions

Phenolic acid standards (vanillic, caffeic, trans-p-coumaric, ferulic acid) and α-tocopherol bought from Sigma-Aldrich (St. Louis, Mo., USA). Lutein (40 mg/capsule) was purchased from GNC (Pittsburgh, Pa., USA) and was used as a reference standard for lutein.

The HPLC of oil solutions were carried out on Waters 2695 HPLC system coupled with a photodiode array detector (PDA) (Waters 2996), an auto-sampler (Waters 717 plus). The HPLC column was a 250×4.6 mm, 5 μm RP C18 column (Waters, Atlantis T3). The mobile phase consisted of A (0.04% acetic acid in deionized water) and B (0.04% acetic acid in methanol). The gradient procedure for HPLC separation is listed in Table 5.

Identification of phenolic acid (vanillic, caffeic, p-coumaric, and ferulic acid), lutein, and a-tocopherol were based on comparing the retention time and UV absorbance of the respective compounds.

TABLE 5
Gradient procedure for chromatographic separation

| | | |
|---|---|---|
| Time | Flow rate | Phase composition |

| (min) | (mL/min) | A/% | B % |
|---|---|---|---|
| 0 | 1 | 100 | 0 |
| 1 | 1 | 100 | 0 |
| 8 | 1 | 90 | 10 |
| 24 | 1 | 75 | 25 |
| 34 | 1 | 55 | 45 |
| 45 | 1 | 45 | 55 |
| 60 | 1 | 0 | 100 |
| 90 | 1 | 0 | 100 |
| 95 | 1 | 100 | 0 |
| 105 | 1 | 100 | 0 |

### Example VIII. SDS-PAGE Analysis of Zein

Running gel for SDS-PAGE was prepared in the following procedures. Bromophenol blue, acrylamide/bis-acrylamide solution (19:1, 40%) and acrylamide/bis-acrylamide solution (29:1, 30%) were purchased from Bio-Rad (Hercules, Calif., USA). Ammonium persulfate (98%) for electrophoresis was purchased from Sigma-Aldrich (St. Louis, Mo., USA). Coomassie brilliant blue G-250 was purchased from AppliChem (Darmstadt, Germany). The separating gel, stacking gel and running buffer were prepared as listed in Table 6.

TABLE 6
Composition of gel and buffer for zein electrophoresis

| | | | Radio of |
|---|---|---|---|
| | Stock solution/100 mL | | stocking solution |
| Separating | A | 1M HCl (48.0 mL) | A:B:C:H₂O = |
| gel | | | 1:3.5:4:1 |
| | | Tris (36.6 g) | |
| | | TEMED (0.23 mL) | |
| | B | 30% (Acr:Bis = 29:1) | |
| | C | Ammonium persulphate | |
| | | (0.14 g) | |
| Stacking | D | 1M HCl (48.0 mL) | C:D:E:F = |
| gel | | Tris (5.98 g) | 0.67:1:1:4 |
| | | TEMED (0.46 mL) | |
| | E | 40% (Acr:Bis = 19:1) | |
| | F | Sucrose (40.0 g) | |

| | |
|---|---|
| Running | Tris-Glycine-SDS Buffer Concentrate for Electrophoresis |
| buffer | Reagent from Sigma-Aldrich (St. Louis, Missouri, USA) |

The electrophoresis of zein was operated with electrophoresis apparatus from Bio-Rad Company (Hercules, Calif., USA). The molecular weight profile of extracted zein from DDGS and whole stillage was measured with a 4% stacking gel and 12% separating gel in an SDS-Tris-Glycine buffer system, following SDS-PAGE method for zein. Briefly, the zein solutions were diluted to 10 g/L by a sample buffer: 125 mM Tris-HCl at pH 7.0, 2% SDS, 10% glycerol, 5% 2-mercaptoethanol and 0.05% bromophenol blue. The protein solutions were centrifuged to remove the precipitation, and 15 μl of the solution was loaded on to the gel. Electrophoresis was performed at 200 V for 60 min. The gel was stained by 0.1% Coomassie brilliant blue solution. Bio-Rad (Hercules, Calif., USA) molecular weight marker ranging from 10 kDa to 200 kDa was used.

### Example IX. HPLC Analytical Procedure for Nucleotides

The HPLC analysis was carried out on a Waters 2695 HPLC system coupled with a photodiode array detector (PDA) (Waters 2996) and auto sampler (Waters 717 plus). The stationery phase was a HPLC column was a 250×4.6 mm, 5 μm C18 column (Atlantis, Waters). The mobile phase A (K₂HPO₄, 0.1M, pH 5.6) was made by dissolving 13.6 g K₂HPO₄ in 1000 mL of de-ionized water and adjusting the pH to 5.6 with 2 M KOH solution. Mobile phase B was 100% of methanol. The solvent gradient sequence was shown in Table 7.

TABLE 7
Gradient procedure for nucleotides HPLC analysis

| | | |
|---|---|---|
| Time | Flow rate | Phase composition |

| (min) | (mL/min) | % A | % B |
|---|---|---|---|
| 0 | 0.5 | 100 | 0 |
| 5 | 0.5 | 100 | 0 |
| 14 | 0.5 | 90 | 10 |
| 15 | 0.5 | 80 | 20 |
| 35 | 0.5 | 80 | 20 |
| 36 | 0.5 | 100 | 0 |
| 50 | 0.5 | 100 | 0 |

In this specification and the appended claims, the singular forms “a,” “an,” and “the” include plural reference, unless the context clearly dictates otherwise. Unless defined otherwise, all technical and scientific terms used herein have the same meaning as commonly understood by one of ordinary skill in the art. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A process for extracting one or more active ingredients from a biomass feedstock, comprising:
contacting a biomass feedstock with an enzyme capable of causing the biomass feedstock to release one or more nucleotides, thereby forming a mixture of a solid phase and an aqueous phase comprising one or more nucleotides;
separating the solid phase from the aqueous phase;
removing water from the aqueous phase to form a first product comprising one or more nucleotides;
contacting the solid phase with a solvent under a condition and for a time sufficient to extract the one or more active ingredients from the solid phase into the solvent, thereby forming a solid residual biomass phase and a liquid phase comprising the solvent and the extracted one or more active ingredients; and
separating the solid residual biomass phase and the liquid phase comprising the solvent and one or more active ingredients.

2. The process of claim 1, further comprising
selectively extract the liquid phase to yield a second product.

3. The process of claim 2, further comprising
selectively extract the liquid phase to yield a third product, and optionally
selectively extract the solid residual biomass phase to yield a fourth product.

4. (canceled)

5. The process of claim 1, wherein the biomass feedstock is selected from whole stillage, thin stillage, Distillers Dried Grains with Solubles (DDGS), syrup, beer, Wet Distillers Grain with Solubles (WDGS), and Modified Wet Distillers Grain with Solubles (Modified WDGS).

6. The process of claim 5, wherein the biomass feedstock comprises a whole stillage or a thin stillage of grains selected from corn, rice, wheat, barley and rye.

7-11. (canceled)

12. The process of claim 1, wherein the enzyme is a nuclease.

13. The process of claim 12, wherein the enzyme is an endonuclease.

14. The process of claim 12, wherein the enzyme is an exonuclease.

15. The process of claim 12, wherein the enzyme is a site-specific nuclease.

16. The process of claim 1, wherein contacting the biomass feedstock with the enzyme is performed at a temperature between about 15° C. to about 70° C. for a time from about 0.5 hour to about 30 hours.

17. The process of claim 1, wherein the first product comprises one or more nucleotides and one or more vitamins.

18. The process of claim 17, wherein the one or more nucleotides comprise adenosine monophosphate (AMP) and guanine monophosphate (GMP) and the one or more vitamins comprise vitamin B family compounds selected from B1, B2, B3, B4, B5 and B6, B9, and choline.

19. (canceled)

20. The process of claim 2, wherein the second product comprises zein.

21. (canceled)

22. The process of claim 3, wherein the third product comprises one or more of lutein, vanillic acid, caffeic acid, p-coumaric acid and ferulic acid, and optionally the fourth product comprises one or more of fibers and proteins.

23. (canceled)

24. The process of claim 1, wherein the solvent comprises an alcohol or an alkyl acetate.

25-31. (canceled)

32. The process of claim 1, wherein separation of a solid phase and a liquid phase is carried out by one or more of: filtration and centrifuge.

33-36. (canceled)

37. A process for extracting one or more active ingredients from a biomass feedstock, comprising:
contacting a biomass feedstock selected from a whole stillage with an enzyme capable of causing the biomass feedstock to release one or more nucleotides, thereby forming a mixture of a solid phase and an aqueous phase comprising one or more nucleotides;
adding lipophilic solvent, preferable ethyl acetate to extract the lipid soluble components from the mixture,
separating oil phase, water phase, and solid phase;
removing water from the aqueous phase to form a first product comprising one or more nucleotides;
removing the ethyl acetate from the oil phase to form a second products a oils with bioactives including tocopherols;
contacting the solid phase with a solvent under a condition and for a time sufficient to extract protein (i.e. zein) from the solid phase into the solvent, thereby forming a solid residual biomass phase and a liquid phase comprising the solvent and zein;
separating the solid residual biomass phase and the liquid phase comprising the solvent and zein;
remove the solvent from the liquid phase to give zein; and
drying the solid residue to give product as spent whole stillage.

38. A process for extracting one or more active ingredients from a biomass feedstock, comprising:
contacting a biomass feedstock selected from a whole stillage with lipophilic solvent, preferable ethyl acetate;
separating lipid phase from the mixture;
removing solvents from the lipid phase to yield oils with bioactives including tocopherols and carotenoids;
contacting the remaining aqueous phase and solid mixture with alcohol to extract proteins (i.e., zein);
separating the liquid phase from the solid phase;
removing the solvent from the liquid phase to yield zein protein;
contacting the solid with water and nuclease to breakdown the RNA into nucleotides;
separating the solid residual biomass phase and the liquid phase comprising water and nucleotides;
remove the water from the liquid phase to give solid containing nucleotides; and
drying the solid residue to give product as spent whole stillage.

39-44. (canceled)

45. A process for extracting one or more nucleotides from a biomass feedstock, comprising:
contacting a biomass feedstock selected from with an enzyme capable of causing the biomass feedstock to release one or more nucleotides, thereby forming a mixture of a solid phase and an aqueous phase comprising one or more nucleotides;
separating the solid phase from the aqueous phase; and
removing water from the aqueous phase to yield one or more nucleotides.

46-54. (canceled)

55. A composition comprising one or more active ingredients extracted by a process according to claim 1.
